(19) United States
(12) Patent Application Publication
(10) Pub. No.: US 2004/0176588 A1
(43) Pub. Date: Sep. 9, 2004

(54) Process for production of sugar oxazoline derivatives

(76) Inventors: Shin-ichiro Shoda, Sendai-shi (JP); Masaya Fujita, Tokyo (JP); Masako Suenaga, Mobara-shi (JP); Kenji Saito, Sendai-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/482,678

(22) PCT Filed: Jun. 28, 2002
(86) PCT No.: PCT/JP02/06575

(30) Foreign Application Priority Data
Jul. 2, 2001 (JP) 2001-201316

(51) Int. Cl. C07H 5/06; C07D 491/02

(52) U.S. Cl. 536/55.3; 548/216

(57) ABSTRACT

A method for production of a sugar oxazoline derivative represented by the following general formula (2), comprising the step of:

reacting an acylamino sugar represented by the following general formula (1) with a metal fluoride;

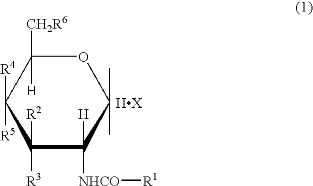

wherein X is selected from F, Cl, Br, and I;

R¹ is selected from H and (CH₂)ₙ—CH₃ wherein n=0 to 5;

R², R³, R⁴, R⁵, and R⁶ are each independently selected from H, N₃, OH protected by a protective group, NH₂ protected by a protective group, and Y—R⁷ wherein Y is O, NH, or S; and R⁷ is a mono- or oligo-saccharide residue, with the proviso that when the residue bears OH, NH₂, or COOH, the groups are protected by protective groups, provided that at least one of R² and R³ and at least one of R⁴ and R⁵ are each H;

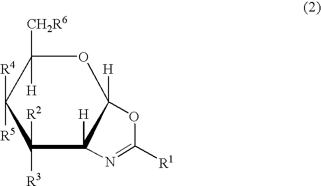

wherein R¹, R², R³, R⁴, R⁵, and R⁶ are the same as those mentioned above.

### TECHNICAL FIELD

The present invention relates to a process for the production of a sugar oxazoline derivative.

### BACKGROUND ART

In recent years, much attention is given to sugar oxazoline derivatives as substrates for glycosylation using sugar-chain related enzymes, and methods for obtaining sugar oxazoline derivatives are investigated. Conventionally, the process for the production of bicyclic sugar oxazoline derivatives, in which a reaction is carried out by adding sodium bicarbonate as an acid trapping agent to an acetonitrile solution of N-acetyl-3,4,6-tri-O-acetyl-α-glucosaminyl chloride using tetraethylammonium chloride as a nucleophilic reagent, has been used (JP 09-3088 A). In this process, however, it is difficult to remove an excessive amount of the reaction agent remaining in the solution after completion of the reaction. Thus, complicated purification procedures are required for obtaining sugar oxazoline derivatives of high purity.

### DISCLOSURE OF THE INVENTION

In recent years, the actions of oligosaccharides, glycosaminoglycans, and so on, which are in vivo materials, are attracting attention in the medical field. Therefore, there is a need of an industrializable, cost effective, and simpler process for the production of sugar oxazoline derivatives to serve as substrates for enzymatic synthesis of the oligosaccharides and glycosaminoglycans.

The inventors of the present invention have made extensive studies with a view to overcome the above-mentioned problems. As a result, they have found out that if a metal fluoride is used as a reaction agent, the fluoride exerts both nucleophilic and acid-trapping actions to expedite the synthesis of sugar oxazoline derivatives, and that the fluoride can be removed easily, thus completing the present invention.

According to the present invention, there is provided a process for production of a sugar oxazoline derivative represented by the following general formula (2), comprising the step of reacting an acylamino sugar represented by the following general formula (1) with a metal fluoride.

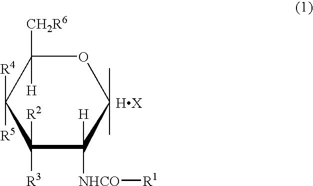

wherein X is selected from F, Cl, Br, and I;

R¹ is selected from H and (CH₂)ₙ—CH₃ wherein n=0 to 5;

R², R³, R⁴, R⁵, and R⁶ are each independently selected from H, N₃, OH protected by a protective group, NH₂ protected by a protective group, and Y—R⁷ wherein Y is O, NH, or S; and R⁷ is a mono- or oligo-saccharide residue, with the proviso that when the residue bears OH, NH₂, or COOH, the groups are protected by the protective groups, provided that at least one of R² and R³ and at least one of R⁴ and R⁵ are each H.

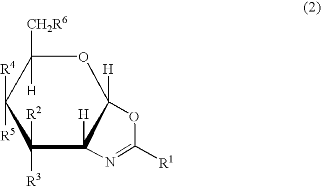

wherein R¹, R², R³, R⁴, R⁵, and R⁶ are the same as those mentioned above.

Further, the present invention is a process for production of a sugar oxazoline derivative represented by the following general formula (3), comprising the steps of: reacting an acylamino sugar represented by the following general formula (1) with a metal fluoride; and removing at least a part of a protective group of the resulting sugar oxazoline derivative.

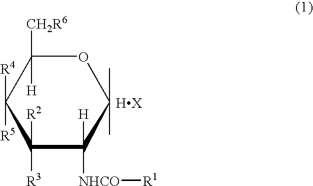

wherein X is selected from F, Cl, Br, and I;

R¹ is selected from H and (CH₂)ₙ—CH₃ wherein n=0 to 5;

R², R³, R⁴, R⁵, and R⁶ are each independently selected from H, N₃, OH protected by a protective group, NH₂ protected by a protective group, and Y—R⁷ wherein Y is O, NH, or S; and R⁷ is a mono- or oligo-saccharide residue, with the proviso that when the residue bears OH, NH₂, or COOH, the groups are protected by protective groups, provided that at least one of R² and R³ and at least one of R⁴ and R⁵ are each H.

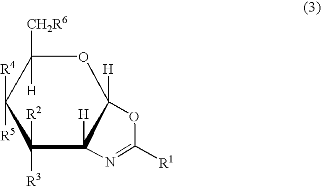

wherein R¹ is the same as that mentioned above,

R², R³, R⁴, R⁵, and R⁶ are each independently selected from H, N₃, OH which may be protected by a protective group, NH₂ which may be protected by a protective group, and Y—R⁷ wherein Y is O, NH, or S; and R⁷ is a mono- or oligo-saccharide residue, with the proviso that when the residue bears OH, NH₂, or COOH, the groups may be protected by protective groups, provided that at least one of R² and R³ and at least one of R⁴ and R⁵ are each H.

In the above-described production processes, it is preferable that the metal fluoride be an alkali metal fluoride.

### BEST MODE FOR CARRYING OUT THE INVENTION

The process for production of the sugar oxazoline derivative represented by the general formula (2) of the present invention comprises the step of reacting an acylamino sugar represented by the general formula (1) with a metal fluoride.

The acylamino sugar represented by the general formula (1) is not particularly restricted by the derivation or origin of its material. It is possible to use acylamino sugars occurring in nature or obtained from animal cells, microorganisms, and so on by genetically engineering with the introduction of protective groups by the conventional method if required, or to use acylamino sugars artificially obtained by chemical synthesis.

X can be selected from the group consisting of fluorine, chlorine, bromine, and iodine, which are classified as halogen. Chlorine is particularly preferable.

R¹ can be selected from H and (CH₂)ₙ—CH₃ wherein n=0 to 5. Among them, H, CH₃, and CH₂CH₃, are preferable and CH₃ is particularly preferable. The presence of these groups will not affect the electronic state of the adjoining amide (—NHCO—) to a large extent.

R², R³, R⁴, R⁵, and R⁶ each can be selected from H, N₃, OH protected by a protective group, NH₂ protected by a protective group, and Y—R⁷ (wherein Y is O, NH, or S; and R⁷ is a mono- or oligo-saccharide residue, with the proviso that when the residue bears OH, NH₂, or COOH, the groups are protected by protective groups) which are given above, provided that at least one of R² and R³ and at least one of R⁴ and R⁵ are each H.

The mono- or oligo-saccharide residue of R⁷ is usually provided as a residue at the 1-position of a monosaccharide or the 1-position of the reducing terminal of an oligosaccharide. The constituent sugar of a mono- or oligo-saccharide may include an amino sugar, a sugar acid, and derivatives thereof. The monosaccharides may preferably include D-glucosamine, D-galactosamine, D-mannosamine, D-galactose, D-glucose, D-mannose, D-glucuronic acid, L-iduronic acid, and derivatives thereof. The oligosaccharides may preferably include: oligosaccharides in which the same monosaccharides are polymerized; glycosaminoglycans each having, as a basic skeleton, a repetitive structure of disaccharide units composed of a uronic acid selected from L-iduronic acid and D-glucuronic acid and a hexosamine selected from D-glucosamine and D-galactosamine, and derivatives thereof. In other words, a dissacharide or glycosaminoglycan in which the sugar residue of the reducing terminal has the structure of monosaccharide represented as the general formula (1) can also be used for the production process of the present invention.

Furthermore, the above derivatives include those obtained by acetylating NH₂ of the constituent sugar of a monosaccharide or oligosaccharide and those obtained by sulfating OH of the same.

In the present invention, oligosaccharides are those referred to oligosaccharides in general constructed of two or more monosaccharides, typically two to twenty several sugars, or two to twenty sugars.

In all cases including the case where a substituent containing the residue of a mono- or oligo-saccharide is selected, the substituents selected for R², R³, R⁴, R⁵, and R⁶ are not substantially involved in the oxazoline-forming reaction of the present invention. Thus, similarly to the ordinary synthetic reaction, if the substituents are functional groups predicted to be highly reactive at the time of the oxazoline-forming reaction of the present invention, there is a need to prevent the functional groups from reacting. For instance, there is considered a process in which, before carrying out the reaction of the present invention, the functional groups are protected by protective groups generally used in the conventional process and then the protective groups are removed from the functional groups after completion of the reaction to thereby obtain a target material. Therefore, the groups predicted to be highly reactive should be protected in the acylamino sugar represented by the general formula (1).

The protective groups are not particularly limited provided that the protective groups do not prevent the reaction of the acylamino sugar represented by the general formula (1) in the presence of a metal fluoride. Specifically, acyl groups such as an acetyl group, a benzoyl group, a methylbenzoyl group, a pivaloyl group, a levulinoyl group, and a t-butyloxycarbonyl group, lower alkyl groups (generally, about 1 to 5 carbon atoms) such as a methyl group and an ethyl group, aralkyl groups such as a benzyl group and a phenethyl group, alkylidene groups such as an isopropylidene group, aralkylidene groups such as a benzylidene group, and aryl groups can be preferably used as the protective groups. Of those, as a protective group for a hydroxyl group or an amino group, an acetyl group can be preferably used. As a protective group for a carboxyl group, a lower alkyl group or an aralkyl group can be preferably used. In general, the protective group can be suitably selected according to the kind of the group to be protected; two or more kinds of the groups may be protected with the same kind of the protective groups, or the same kind of the groups may be protected with two or more kinds of the protective groups.

Preferable examples of acylamino sugar to be used in the present invention include the following examples 1 to 9.

Example 1: acylamino sugar represented by the general formula (1) wherein X: Cl, R¹: CH₃, R 2: OCOCH₃, R³: H, R⁴: H, R⁵: OCOCH₃, and R⁶: OCOCH₃.

Example 2: acylamino sugar represented by the general formula (1) wherein X: Cl, R¹: CH₃, R 2: OCOCH₃, R³: H, R⁴ OCOCH₃, R⁵: H, and R⁶: OCOCH₃.

Example 3: acylamino sugar represented by the general formula (1) wherein X: Cl, R¹: CH₃, R 2: OCOCH₃, R³: H, R⁴: H, R⁵: O-β-D-galactose (wherein the OH is protected by an acetyl group), and R⁶: OCOCH₃.

Example 4: acylamino sugar represented by the general formula (1) wherein X: Cl, R¹: CH₃, R²: O-β-D-glucuronic acid (wherein the OH is protected by an acetyl group and the COOH is protected by a benzyl group), R³: H, R⁴: H, R⁵: OCOCH₃, and R⁶: OCOCH₃.

Example 5: acylamino sugar represented by the general formula (1) wherein X: Cl, R¹: CH₃, R²: O-β-D-glucuronic acid (wherein the OH is protected by an acetyl group, and the COOH is protected by a benzyl group), R³: H, R⁴: OCOCH₃, R⁵: H, and R⁶: OCOCH₃.

Example 6: acylamino sugar represented by the general formula (1) wherein X: Cl, R¹: CH₃, R²: O-α-L-iduronic acid (wherein the OH is protected by an acetyl group, and the COOH is protected by a benzyl group), R³: H, R⁴: OCOCH₃, R⁵: H, and R⁶: OCOCH₃.

Example 7: acylamino sugar represented by the general formula (1) wherein X: Cl, R¹: CH₃, R²: OCOCH₃, R³: H, R⁴: H, R⁵: O-β-D-glucosamine (wherein the OH and NH₂ are both protected by acetyl groups), and R⁶: OCOCH₃.

Example 8: acylamino sugar represented by the general formula (1) wherein X: Cl, R¹: CH₃, R²: OCOCH₃, R³: H, R⁴: H, R⁵: O-β-N-acetyl-D-glucosamine (wherein the OH is protected by an acetyl group), and R⁶: OCOCH₃.

Example 9: acylamino sugar represented by the general formula (1) wherein X: Cl, R¹: CH₃, R²: OCOCH₃, R³: H, R⁴: H, R⁵: a sugar residue (wherein unsulfated OH and NH₂ are protected by acetyl groups and the COOH is protected by a benzyl group) having an uronic acid residue (β-D-glucuronic acid residue or α-L-iduronic acid residue) on the reducing terminal side of O-heparin, and R⁶: OCOCH₃ (heparin including a glucosamine residue having Cl at its 1-position and an acetylated amino group at its 2-position, where other unsulfated hydroxyl groups and amino groups are acetylated, and a carboxyl group is benzylated).

Furthermore, the acylamino sugar indicated in Example 1 is one in which the OH at each of 3-, 4-, and 6-positions of N-acetyl-D-glucosamine, which is a constituent sugar of chitin, is protected by an acetyl group and the OH at the 1-position is replaced with a chlorine atom. The acylamino sugar indicated in Example 4 is one in which each OH of a constituent disaccharide unit of hyaluronic acid constructed of an uronic acid and a hexosamine (at 4- and 6-positions of the hexosamine and 2-, 3-, and 4-positions of the uronic acid) is protected by an acetyl group, the carboxyl group of an uronic acid residue is protected by a benzyl group, and the OH at the 1-position of a hexosamine residue is replaced with a chlorine atom. The acylamino sugar indicated in Example 5 is one in which each OH of a constituent disaccharide unit of chondroitin constructed of an uronic acid and a hexosamine (at 4- and 6-positions of the hexosamine and 2-, 3-, and 4-positions of the uronic acid) is protected by an acetyl group, the carboxyl group of an uronic acid residue is protected by a benzyl group, and the OH at the 1-position of a hexosamine residue is replaced with a chlorine atom. The acylamino sugar indicated in Example 6 is one in which each OH of a constituent disaccharide unit of dermatan sulfate constructed of an uronic acid and a hexosamine (at 4- and 6-positions of the hexosamine and 2-, 3-, and 4-positions of the uronic acid) is protected by an acetyl group, the carboxyl group of an uronic acid residue is protected by a benzyl group, and the OH at the 1-position of a hexosamine residue is replaced with a chlorine atom.

The metal fluoride to be used in the production process of the present invention is not particularly limited, but is preferably an alkaline metal fluoride, and more preferably sodium fluoride, potassium fluoride, rubidium fluoride, cesium fluoride, or the like. At the time of the production on an industrial scale, sodium fluoride and potassium fluoride are particularly preferable in consideration of handling, price, and so on as a reagent.

The metal fluoride may be held on an inorganic solid carrier likewise with the conventional process. Examples of the inorganic solid carrier include alumina, silica gel, magnesium oxide, molecular sieves (e.g., Linde 4A (trade name)), clay (e.g., montmorillonite), and diatomaceous earth (e.g., Celite (trade name). Among them, alumina is particularly preferable.

The conditions for reaction between the acylamino sugar represented by the general formula (1) and the metal fluoride are not specifically limited provided that a bicyclic sugar oxazoline derivative can be generated by the reaction between the acylamino sugar and the metal fluoride, which are used in the present invention, and thus the conditions can be suitably set by a person skilled in the art.

A solvent to be used in the reaction is not particularly limited provided that the solvent do not react with the acylamino sugar and the metal fluoride to be used and is capable of dissolving the acylamino sugar therein. As the solvent, acetonitrile (CH₃CN), dimethyl sulfoxide (DMSO), dimethylformamide (DMF), tetrahydrofuran, xylene, and the like are preferable, and among them, CH₃CN, DMSO, and DMF are more preferable. In particular, CH₃CN is preferable.

The amount of metal fluoride to be used and the reaction conditions such as a reaction temperature and a reaction time can be suitably set depending on the amount of acylamino sugar to be used and so on. A molar ratio between the acylamino sugar and the metal fluoride is preferably 1:2 to 1:30, more preferably 1:10. The reaction temperature is preferably from room temperature to a boiling point of the solvent, more preferably from 30 to 60° C. Furthermore, in the case of carrying out the reaction at the boiling point of the solvent to be used, a reflux condenser or the like may be used. The reaction time is preferably 0.5 hours to three days.

At the time of setting the reaction conditions, it is preferable to confirm the completion of the reaction. A concrete example of a method for confirming the completion of the reaction is thin-layer chromatography.

A reaction between the acylamino sugar represented by the general formula (1) and the metal fluoride is preferably performed under an atmosphere of argon, nitrogen, or the like to avoid the reaction with water.

As a method for purifying a sugar oxazoline derivative as a target material from a reaction-completed solution, any of the purification processes conventionally performed may be suitably selected and used. For example, after removal of an insoluble matter from the reaction-completed solution, a water-soluble material being dissolved in the reaction-completed solution was removed by a liquid-separating operation, followed by purifying with silica-gel chromatography, recrystallization, or the like.

A method for removing an insoluble matter from the reaction-completed solution has only to divide an insoluble matter (solid) and a solution (liquid) and thus may be a method including a filtration process using a glass filter, celite, or the like, which is generally used in the art.

The process for production of the sugar oxazoline derivative represented by the general formula (3) is characterized in that the sugar oxazoline derivative represented by the general formula (2) is obtained by the above method and at least a part of the protective group of the obtained sugar oxazoline derivative is removed. In this process, the sugar oxazoline derivative represented by the general formula (3) can be obtained, in which at least a part of the protective group of the sugar oxazoline derivative represented by the general formula (2) is removed. The protective group can be removed by the conventional method.

The sugar oxazoline derivative obtained by the production process of the present invention may be used as a substrate for the synthesis of a polymer using the ring-opening polymerization of an oxazoline ring. In particular, the sugar oxazoline derivative obtained according to the production process of the present invention using as an acylamino sugar one in which at least one of R² to R⁶ is a mono- or oligo-saccharide (a disaccharide or glycosaminoglycan having a specific structure on the reducing terminal) may be used as a substrate for synthesizing glycosaminoglycans using an enzyme-catalyzed polyaddition reaction.

For instance, a bicyclic sugar oxazoline derivative having an oxazoline ring on the reducing terminal of a lactosamine chain containing one or more basic skeletons each constructed of a disaccharide of lactosamine is obtained by the production process of the present invention. Then, keratanase is acted on the resulting derivative provided as a substrate to allow the elongation of the lactosamine chain. Likewise, hyaluronidase is acted on, as a substrate, a bicyclic sugar oxazoline derivative having an oxazoline ring on the reducing terminal of a hyaluronic acid chain having one or more basic skeletons each constructed of a disaccharide of hyaluronic acid, which can be obtained by the production process of the present invention, to allow the elongation of the hyaluronic acid chain. In addition, it is also conceived that chitinase is acted on, as a substrate, glucosamine having an oxazoline ring or a disaccharide in which N-acetylglucosamine or glucosamine is bonded to the non-reducing terminal thereof obtained by the production process of the present invention to allow chitin or chitosan to be obtained.

### EXAMPLES

Hereinafter, the present invention will be described more concretely on the basis of examples.

### Example 1

### Synthesis of Bicyclic Oxazoline Using Metal Fluoride

Under each of the reaction conditions described in Table 1 below, the synthesis was carried out by the following procedures.

Under an argon atmosphere, 75 ml of an acetonitrile solution of 1.8 g (5 mmol) N-acetyl-3,4,6-tri-O-acetyl-α-glucosaminyl chloride or 75 ml of a dimethylformamide solution of 1.8 g. (5 mmol) N-acetyl-3,4,6-tri-O-acetyl-α-glucosaminyl chloride was added to potassium fluoride or cesium fluoride to carry out a reaction under reflux. The completion of the reaction was confirmed by thin-layer chromatography, followed by filtration with a glass filter G4. Then, the resultant filtrate was condensed with an evaporator and was diluted with an excess amount of chloroform. Until the pH of the solution became neutral, a liquid-separating operation was performed with a sodium bicarbonate saturated solution and subsequently with cold water. The collected organic layer was dried on anhydrous sodium sulfate. Then, the sodium sulfate was removed by filtration and the filtrate was condensed with an evaporator. The obtained residue was purified by silica-gel flash chromatography (gel: Silika gel 60 manufactured by Merck Co., Ltd., 0.040 to 0.063 mm in particle size, developing solvent: hexane/ethyl acetate=2/3 (volume ratio)). The solution after the development was further condensed with the evaporator and was dried under reduced pressure, resulting in 2-methyl (3,4,6-tri-O-acetyl-1,2-dideoxy-α-glucopyrano) [2,1-d]-2-oxazoline in the form of yellow syrup. The yield thereof is shown in Table 1.

TABLE 1

| | | | Reaction | Reaction | |
|---|---|---|---|---|---|
| Metal | | | temperature | time | |
| fluoride | Equivalent*¹ | Solvent | (° C.) | (time) | Yield (%) |
| KF | 1 | CH₃CN | 60 | 24 | 71.4 |
| | 5 | CH₃CN | 60 | 78 | 81.3 |
| | 10 | CH₃CN | 60 | 88 | 78.7 |
| | 10 | DMF | 60 | 24 | 66.2 |
| CsF | 1 | CH₃CN | Room temp. | 24 | 9.5 |
| | 5 | CH₃CN | Room temp. | 24 | 10.0 |
| | 10 | CH₃CN | 60 | 1 | 34.7 |
| | 10 | DMF | Room temp. | 24 | 18.5 |

*¹Equivalent to N-acetyl-3,4,6-tri-O-acetyl-α-glucosaminyl chloride (10 equivalents of potassium fluoride is 2.9 g and 10 equivalents of cesium fluoride is 7.6 g)

The NMR data of the resulting product (2-methyl (3,4,6-tri-O-acetyl-1,2-dideoxy-α-glucopyrano) [2,1-d]-2-oxazoline) was as follows:

¹H NMR (250 MHz, CDCl₃): 5.95 (1H, d, J_{1,2}=7.34 Hz, anomeric proton), 2.0-2.1 (12H, m, methyl proton of acetate and methyl proton of oxazoline); and

¹³C NMR (62.9 MHz, CDCl₃): 99.4 (C-1), 20.7-20.9 (3C, methyl C of acetate), 13.9 (methyl C of oxazoline), 169.2-170.6 (3C, carbonyl C of acetyl), 166.7 (C of oxazoline ring: O—C═N).

### INDUSTRIAL APPLICABILITY

According to the present invention, there is provided a mass-productive, cost effective, and simpler process for the production of sugar oxazoline derivatives.

What is claimed is:

1. A method for production of a sugar oxazoline derivative represented by the following general formula (2), comprising the step of:
reacting an acylamino sugar represented by the following general formula (1) with a metal fluoride; 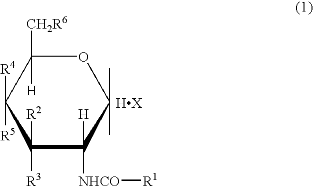
wherein X is selected from F, Cl, Br, and I;
R¹ is selected from H and (CH₂)ₙ—CH₃ wherein n=0 to 5;
R², R³, R⁴, R⁵, and R⁶ are each independently selected from H, N₃, OH protected by a protective group, NH₂ protected by a protective group, and Y—R⁷ wherein Y is O, NH, or S; and
R⁷ is a mono- or oligo-saccharide residue, with the proviso that when the residue bears OH, NH₂, or COOH, the groups are protected by protective groups, provided that at least one of R² and R³ and at least one of R⁴ and R⁵ are each H; 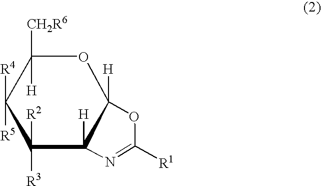
wherein R¹, R², R³, R⁴, R⁵, and R⁶ are the same as those mentioned above.

2. The method for production according to claim 1, wherein the metal fluoride is an alkali metal fluoride.

3. A method for production of a sugar oxazoline derivative represented by the following general formula (3), comprising the steps of:
reacting an acylamino sugar represented by the following general formula (1) with a metal fluoride; and
removing at least a part of a protective group of the resulting sugar oxazoline derivative; 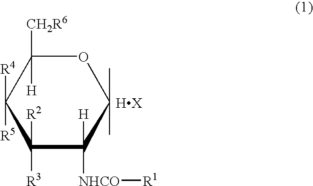
wherein X is selected from F, Cl, Br, and I;
R¹ is selected from H and (CH₂)ₙ—CH₃ wherein n=0 to 5;
R², R³, R⁴, R⁵, and R⁶ are each independently selected from H, N₃, OH protected by a protective group, NH₂ protected by a protective group, and Y—R⁷ wherein Y is O, NH, or S; and
R⁷ is a mono- or oligo-saccharide residue, with the proviso that when the residue bears OH, NH₂, or COOH, the groups are protected by the protective groups, provided that at least one of R² and R³ and at least one of R⁴ and R⁵ are each H; 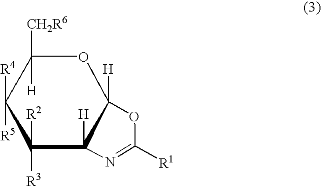
wherein R¹ is the same as that mentioned above,
R², R³, R⁴, R⁵, and R⁶ are each independently selected from H, N₃, OH which may be protected by a protective group, NH₂ which may be protected by a protective group, and Y—R⁷ Wherein Y is O, NH, or S; and R⁷ is a mono- or oligo-saccharide residue, with the proviso that when the residue bears OH, NH₂, or COOH, the groups may be protected by the protective groups, provided that at least one of R² and R³ and at least one of R⁴ and R⁵ are each H.

4. The method for production according to claim 3, wherein the metal fluoride is an alkali metal fluoride.
